(19) United States
(12) Patent Application Publication
(10) Pub. No.: US 2011/0081313 A1
(43) Pub. Date: Apr. 7, 2011

(54) MECHANISM OF ACTION OF PRIMARY CELL DERIVED BIOLOGIC

(75) Inventor: John W. Hadden, Cold Spring Harbor, NY (US)

(73) Assignee: IRx Therapeutics, Inc.

(21) Appl. No.: 12/995,054

(22) PCT Filed: May 29, 2009
(86) PCT No.: PCT/US09/45550

§ 371 Date: Nov. 29, 2010

Related U.S. Application Data
(60) Provisional application No. 61/056,925, filed on May 29, 2008.

(51) Int. Cl. A61K 38/20; G01N 33/574; A61P 35/00; A61P 37/04

(52) U.S. Cl. 424/85.2; 435/7.1

(57) ABSTRACT

A method of treating an immune target that is suppressing the immune system and restoring the immune system, including the steps of administering an effective amount of a primary cell derived biologic, modifying populations of B and T cells in blood, activating regional lymph nodes, infiltrating an area adjacent to an immune target with T helper and B cells, infiltrating the immune target with T killer cells and macrophages, and treating the immune target and restoring the immune system. A method of inducing immunization in a patient. A method of destroying a tumor. A method of predicting a favorable treatment outcome to cancer treatment. A method of immune prophylaxis. A method of immune restoration. A method of treating a tumor. A method of preventing tumor escape.

### BACKGROUND OF THE INVENTION

(1) Field of the Invention

The present invention relates to therapy of the immune system. In particular, the present invention relates the mechanism of action of a primary cell derived biologic on the immune system.

(2) Description of Related Art

Since toxin-induced tumor regressions of human cancer were performed by William Coley early in the 20^{th} century, cancer therapists have employed hundreds of different immune therapies with only relatively rare clinical responses. Because there were little or no insights into the cause of these failures, no consistent mechanism of action emerged. In order to establish a clear mechanism of action, a therapy needed to be devised which could consistently produce a response and which could then be dissected.

Head and neck squamous cell cancer (H&NSCC) offers a good model since much is known about the immune defects seen in these patients. They include, to name a few, (Whiteside, 2001; Hadden, 1995): 1) T lymphocyte energy and depletion induced by tumor and host-mediated mechanism including prostaglandins, T regs, myeloid suppressor cells, antigen-antibody complexes, cytokines such as IL-10, etc.; 2) monocyte/macrophage functional defects with evidence of suppressor and inflammatory changes (Mantovani, 2002); and 3) dendritic cell (DC) defects characterized by sinus histiocytosis (SH) (Dunn, 2005).

Effective therapeutic efforts were needed to reverse the defects. An extensive review of the literature (Hadden, 1995) and a series of pre-clinical experiments resulted in the primary cell derived biologic (also known as IRX-2) protocol. The IRX-2 protocol, shown in FIG. 1, employs an initial dose of low dose cyclophosphamide (CY) (300 mg/m²) by intravenous infusion to reverse suppression by T regs lymphocytes and perhaps other forms of suppressors. The CY is followed by 10-20 daily injections of IRX-2 at the base of the skull to feed into the jugular chains of lymph nodes regional to the cancer. These nodal sites are where an immunization is known to occur (Maass, 1995).

IRX-2 was thought to act via increasing T lymphocyte number and function. Recent evidence indicates that reversal of tumor-induced apoptosis is a major mechanism, as disclosed in U.S. Provisional Patent Application No. 60/990,759 to Signorelli, et al. Indomethacin (INDO) was administered daily for approximately 21 days to block prostaglandin production by tumor and monocyte/macrophages, a known cancer related suppression mechanism. Zinc was also administered as another aspect of the immunorestorative component of the strategy (Hadden, 1995).

Also, at the time the protocol was developed, the critical role played by dendritic cells as presenters of tumor antigen to T cells was unknown and it was also unknown that SH reflected a DC defect. Mechanism of action studies disclosed in U.S. Pat. Nos. 6,977,072 and 7,153,499 to Applicant made it clear that the IRX-2 protocol reverses this DC defect and produces changes in regional lymph nodes which reflect a potent immunization (Meneses, 2003). More specifically, these patents disclose a method of inducing the production of naïve T cells and restoring T cell immunity by administration of IRX-2, which preferably includes the cytokines IL-2, IL-6, IL-8, INF-γ, and TNF-α. This was one of the first showings that adult humans can generate naïve T cells through molecular therapy. It was the presence of naïve T cells that could present to antigen that allowed for immunity to be restored.

The mechanistic hypothesis that underpins IRX-2 is similar to that of a therapeutic cancer vaccine, although no antigen is required to be injected. When administered into the neck, the agent is thought to act in the cervical lymph node chain directly on DCs to foster maturation and their subsequent ability to present endogenous tumor antigen to naïve T cells.

Non-clinical data on IRX-2's mechanism of action has shown that the agent effectively stimulates and activates human monocyte-derived DCs (Egan, 2007). IRX-2 treatment of immature DCs increased expression of CD83 and CCR7 (markers for maturation and lymph node migration, respectively), as well as differentiation molecules that are important for antigen presentation to naïve T cells. Additionally, IRX-2 induces CD48, CD54, and CD86, which are co-stimulatory receptors that are critical for activation of naïve T cells. Functional changes in IRX-2 treated DCs included an increase in antigen presentation and T cell activity. Taken collectively, IRX-2 treatment of immature DC drives T-morphologic, phenotypic, and functional changes that are consistent with the development of mature and activated DCs that are able to effectively stimulate naïve T cells.

In contrast to defined antigen-based therapeutic cancer vaccines where antigen-specific reactivity can be measured, rejection antigens have not been discovered in H&NSCC, thus limiting the ability to measure antigen-specific reactivity after IRX-2 therapy.

While IRX-2 was shown to increase T lymphocyte function, generate new immature T cells, and prevent apoptosis of those T cells once generated, it was not known what the function of the T cells were after presentation of antigen. The exact mechanism by which the T cells treat tumors was neither expressly nor inherently disclosed in the prior art. Furthermore, while IRX-2 was shown to be effective in the mechanisms described above during cancer treatment, there has been no evidence that IRX-2 provides the same mechanism of action in other instances of immune suppression besides cancer. Not only have individual cytokines not been able to completely restore each part of the immune system, other therapeutics including multiple cytokines have not been able to do this as well. For example, MULTIKINE (Cel-Sci) is effective only on the tumor itself, affecting the cell cycle of the tumor cells, and has shown no evidence of affecting the immune system.

In essence, the earlier work of Applicant described the mechanism of action of the primary cell derived biologic with respect to several specific levels of affecting the immune system. Presented herein is evidence of another level of affecting the immune system, i.e. the affect of the primary cell derived biologic on the survival of lymphocytes. The data herein shows that the primary cell derived biologic has a corrective and positive effect on each level of the immune system, i.e. each arm of the immune system. Compositions of the prior art are directed to a single arm of the immune system.

Therefore, there is a need for a composition that can effectively target each arm of the immune system to restore the immune system and provide a complete mechanism of action against immune suppression.

### BRIEF SUMMARY OF THE INVENTION

The present invention provides for a method of treating an immune target that is suppressing the immune system (such as a solid tumor, bacterial infection, or disease such as HIV) and restoring the immune system, including the steps of administering an effective amount of a primary cell derived biologic, modifying populations of B and T cells in blood, activating regional lymph nodes, infiltrating an area adjacent to an immune target with T helper and B cells, infiltrating the immune target with killer T cells and macrophages, and treating the immune target and restoring the immune system.

The present invention also provides for a method of inducing immunization in a patient, including the steps of administering an effective amount of a primary cell derived biologic, detecting a change in T and B cells, and inducing immunization in a patient.

The present invention also provides for a method of destroying a tumor, including the steps of administering an effective amount of a primary cell derived biologic, maturing immature dendritic cells, activating naïve T cells, the resulting mature dendritic cells stimulating the naïve T cells, differentiating the naïve T cells into killer T cells, directing killer T cells to a tumor, and destroying the tumor.

The present invention provides for a method of predicting a favorable treatment outcome to cancer treatment, including the steps of administering an effective amount of a primary cell derived biologic, detecting an increase peritumorally of T helper and B cells and intratumorally of T killer cells and macrophages, and predicting a favorable treatment outcome to cancer treatment.

The present invention provides for a method of immune prophylaxis, including the steps of administering an effective amount of a primary cell derived biologic, and preventing immune suppression.

The present invention further provides for a method of immune restoration, including the steps of administering an effective amount of a primary cell derived biologic, and restoring the immune system of a patient.

The present invention provides for a method of treating a tumor, including the steps of administering an effective amount of a primary cell derived biologic, modifying populations of B and T cells in blood, activating regional lymph nodes, peritumorally infiltrating the tumor with T helper and B cells, intratumorally infiltrating the tumor with T killer cells and macrophages, and treating the tumor.

The present invention also provides for a method of preventing tumor escape, including the steps of administering an effective amount of a primary cell derived biologic, producing an immune regression of a tumor by modifying populations of B and T cells in blood, activating regional lymph nodes, peritumorally infiltrating the tumor with T helper and B cells, intratumorally infiltrating the tumor with T killer cells and macrophages, and preventing tumor escape.

### BRIEF DESCRIPTION OF THE SEVERAL VIEWS OF THE DRAWINGS

Other advantages of the present invention will be readily appreciated as the same becomes better understood by reference to the following detailed description when considered in connection with the accompanying drawings wherein:

FIG. 1 is a display of the IRX-2 protocol;

FIG. 2 is a graph of in vivo dose response for IRX-2;

FIG. 3 is a graph of percentage of survival in four groups of patients;

FIG. 4 is a graph of median percentage of lymphocyte infiltration in four groups of patients;

FIG. 5 is a photograph of H&E staining for lymphocytes;

FIG. 6 is a photograph of H&E staining for lymphocyte infiltration;

FIG. 7A is a graph of lymphoid infiltration density in responders, and

FIG. 7B is a graph of lymphoid infiltration density in non-responders;

FIG. 8 is a graph of location of intratumoral/peritumoral lymphocyte infiltrates;

FIG. 9 is a photograph of IHC staining for CD45RO+ memory T cells; and

FIG. 10 is a photograph of fused FDG PET/CT scan images at day 0 and day 21.

### DETAILED DESCRIPTION OF THE INVENTION

In general, the present invention is directed to the mechanism of action of IRX-2 both with respect to tumors and the immune system in general and provides for a method of treating an immune target by the administration of a primary cell derived biologic. The primary cell derived biologic produces an immune rejection of the immune target, as further described below.

As used herein, the term “immune target” refers to any biological condition that results in a suppression of the immune system or disease that results in immune suppression. The immune target is an otherwise antigenic target that the immune system is nonresponsive to due to suppression. In the present invention, the immune target is “targeted” by the primary cell derived biologic which reverses the immune suppression and restores the immune system to a normal function. The immune target can be caused by genetic defects in the components of the immune system (intrinsic, or primary immune deficiencies). The immune target can also be caused by extrinsic factors (secondary immune deficiencies). For example, the immune target can be caused by a disease such as AIDS or HIV, irradiation (radiotherapy), chemotherapy, malnutrition, burns, infections, and especially cancer (tumors).

As used herein, “apoptosis” refers to cell death. Apoptosis (Type I cell-death) is a type of programmed cell death that occurs for various reasons such as stress, infection, or damage. Apoptosis of lymphocytes can be induced by a variety of phenomena, such as, but not limited to cancer related therapies (chemotherapy, radiation), and tumors themselves producing apoptosis-inducing factors.

As used herein, “lymphocytes” refers to a white blood cell present in the immune system and includes large granular lymphocytes (natural killer (NK) cells) and small lymphocytes (T cells and B cells).

A “primary cell derived biologic”, as used herein, is a combination of cytokines, preferably natural and non-recombinant cytokines, also previously known as a natural cytokine mixture (NCM). Preferably, the primary cell derived biologic is IRX-2 as described below, and the two terms can be used interchangeably throughout this application without derivation from the intended meaning.

“IRX-2” is a leukocyte-derived, natural primary cell derived biologic produced by purified human white blood cells (mononuclear cells) stimulated by phytohemagglutinin (PHA) and ciprofloxacin (CIPRO). The major active components are interleukin 1β (IL-1β), interleukin 2 (IL-2), interleukin 6 (IL-6), interleukin 8 (IL-8), tumor necrosis factor α (TNF-α), and γ-interferon (IFN-γ). Preferably, the IRX-2 used in the present invention includes these six critical cytokines. IRX-2 has also previously been referred to as an “NCM”, a natural cytokine mixture, defined and set forth in U.S. Pat. Nos. 6,977,072 and 7,153,499.

Briefly, IRX-2 is prepared in the continuous presence of a 4-aminoquinolone antibiotic and with the continuous or pulsed presence of a mitogen, which in the preferred embodiment is PHA. However, other mitogens can also be used. The IRX-2 produced for administration to patients contains a concentration of IL-1β that ranges from 60-6,000 pcg/mL, more preferably, from 150-1,800 pcg/mL; a concentration of IL-2 that ranges from 600-60,000 pcg/mL, more preferably, from 3,000-12,000 pcg/mL, and concentrations of IFN-γ and TNF-α that range from 200-20,000 pcg/mL, more preferably, from 1,000-4,000 pcg/mL.

IRX-2 can also contain a concentration of IL-6 that ranges from 60-6,000 pcg/mL, more preferably, from 300-2,000 pcg/mL; a concentration of IL-8 that ranges from 6000-600,000 pcg/mL, more preferably from 20,000-180,000 pcg/mL; a concentration of TNF-α that ranges from 200-20,000 pcg/ml, more preferably, from 1,000-4,000 pcg/mL. Recombinant, natural or pegylated cytokines can be used or IRX-2 can include a mixture of recombinant, natural or pegylated cytokines. The IRX-2 of the present invention can further include other recombinant, natural or pegylated cytokines such as IL-7, IL-12, IL-15, GM-CSF (at a concentration that ranges from 100-10,000 pcg/mL, more preferably from 500-2,000 pcg/mL), and G-CSF. The method of making IRX-2 is disclosed in the above cited patents as well as in U.S. Provisional Patent Application No. 61/044,674.

Other compounds can also be administered along with IRX-2 such as chemical inhibitors, non-steroidal anti-inflammatory drugs (NSAIDS), and combinations thereof. The chemical inhibitor can be any chemotherapeutic agent that is not immunosuppressive (preferably used at low doses) and that has immunomodulatory effects so as to increase immunity and/or an immune response, e.g., by inhibiting immune suppression or suppressor mechanisms in the body. According to a preferred embodiment, the chemical inhibitor is an anti-neoplastic agent, including but not limited to alkylating agents, antimetabolites and antibiotics. The chemical inhibitor can also be an immunomodulating agent such as thalidomide. The chemical inhibitor can also be in a salt or other complex form. Preferably, the chemical inhibitor is the alkylating agent cyclophosphamide (CY). The NSAID is preferably indomethacin (INDO), which is both a CoxI and CoxII inhibitor. The NSAID can also be ibuprofen or CoxII inhibitors such as celecoxib and rofecoxib, or combinations thereof. Also, endogenous antigens (i.e. those already within the body) and exogenous antigens can be administered with IRX-2.

As used herein, “effective amount” refers to an amount of IRX-2 that is needed to achieve the desired result of the present invention, namely, treating an immune target and performing the functions further described below. One skilled in the art can determine the effective amount of the IRX-2 that should be given to a particular patient, with the various concentrations of the components as described above.

The present invention is directed to a method of treating an immune target that is suppressing the immune system and restoring the immune system, including the steps of administering an effective amount of a primary cell derived biologic, modifying populations of B and T cells in blood, activating regional lymph nodes, infiltrating an area adjacent to an immune target with T helper and B cells, infiltrating the immune target with killer T cells and macrophages, and treating the immune target and restoring the immune system. These steps together produce evidence of immune rejection of the immune target. In other words, each of these steps is evidence that the immune system has recognized that the immune target must be destroyed as well as evidence that the immune system has been restored to function normally (or at a higher level than previously in a disease or immune suppressed state).

The primary cell derived biologic, i.e. IRX-2, administered is preferably as described above. A chemical inhibitor, low dose cyclophosphamide is preferably administered prior to administering the IRX-2, which reverses suppression by T regs lymphocytes. An NSAID (preferably indomethacin) and zinc can also be administered daily during the IRX-2 regimen. Dosing of IRX-2 is further described below.

The populations of B and T cells can be up-regulated or down-regulated due to IRX-2 administration. The populations of B and T cells in the blood that are modified are more specifically populations of naïve T cells and early memory T cells. The populations of naïve T cells that are modified are CD3+, CD45RA+, and CCR7+. This is accomplished by differentiating the naïve T cells into memory and effector T cells, which is a time dependent process. The central memory T cells are also caused to exit the bloodstream and migrate to draining lymph nodes. In other words, the modification of levels of naïve T cells is the result of the naïve T cells differentiating into more advanced forms of T cells that can effectively attack the immune target. The populations of B cells in the blood are also modified because the B cells are recruited into lymph nodes, exposed to antigen, migrate to the immune target, and attack the immune target. More specifically, the B cells attack the immune target by producing antibodies and/or supporting antibody-dependent cellular cytotoxicity.

The regional lymph nodes are activated by enlarging the regional lymph nodes, replenishing lymphocytes, and reversing sinus histiocytosis. Immunization to antigen to the immune target occurs in the regional lymph nodes.

Infiltration of the area adjacent to the immune target occurs with CD45RA+, CD3+, and CD4+ T lymphocytes and CD20+ B lymphocytes. The area adjacent to the immune target can range from the surface of the immune target itself to a distance past the surface. Infiltration of the immune target itself, i.e. directly within the immune target, occurs with CD45RO+, CD3+, and CD8+ lymphocytes (i.e. killer T cells) and CD68+ macrophages. Each of these infiltration processes would contribute t providing producing humoral (mediated by antibodies) as well as cellular (mediated by cells) immunity.

Various other procedures can be performed in combination with the IRX-2 administration in each of the methods of the present invention to further enhance therapy such as, but not limited to, surgery, radiotherapy, chemotherapy, or combinations thereof. For example, IRX-2 administration before radiotherapy or chemotherapy (cytodestructive processes) improves the results of these processes because IRX-2 acts as a cytoprotectant by protecting T lymphocytes from apoptosis.

More specifically, there are several ways in which the T cells are protected from apoptosis. The expression of anti-apoptotic signaling molecules are upregulated (i.e. JAK-3 and phosphor-Akt) and the expression of pro-apoptotic molecules are downregulated (i.e. SOCS-2). Overall, caspase activation in CD8+ and CD4+ T lymphocytes is decreased and cFLIP expression is increased. Inhibition of the PI3K/Akt survival pathway is counteracted by IRX-2. The T cells are protected from both extrinsic apoptosis (MV-induced and CH-11Ab-induced apoptosis) and intrinsic mitochondrial apoptosis. Each of these steps of protection are further described in U.S. Provisional Patent Application No. 60/990,759 to Signorelli, et al.

The present invention also provides for a method of inducing immunization in a patient, including the steps of administering an effective amount of the primary cell derived biologic, detecting a change in T and B cells, and inducing immunization in a patient. Administration of the primary cell derived biologic is described above and further below. The changes in the T and B cells are as described above, i.e. a modification in levels of T cells and B cells in blood because they are differentiating or moving to other areas. This movement in the T and B cells is evidence that immunization has been induced in a patient.

A method of destroying a tumor is provided, including the steps of administering an effective amount of the primary cell derived biologic, maturing immature dendritic cells, activating naïve T cells, the resulting mature dendritic cells stimulating the naïve T cells, differentiating the naïve T cells into killer T cells, directing killer T cells to a tumor, and destroying the tumor. The primary cell derived biologic causes maturation of dendritic cells as well as inducing the production of naïve T cells as described in U.S. Pat. Nos. 6,977,072 and 7,153,499. The mature dendritic cells can then present antigen to the naïve T cells so that the naïve T cells can become activated. As evidenced herein, the naïve T cells can now differentiate into killer T cells and become directed to a tumor so that the tumor can be destroyed.

The present invention also provides a method of predicting a favorable treatment outcome to cancer treatment, including the steps of administering an effective amount of the primary cell derived biologic, detecting an increase peritumorally of T helper and B cells and intratumorally of T killer cells and macrophages, and predicting a favorable treatment outcome to cancer treatment. More specifically, an increase is detected peritumorally of CD45RA+, CD3+, and CD4+ T lymphocytes and CD20+ B lymphocytes and intratumorally of CD45RO+, CD3+, and CD8+ lymphocytes and CD68+ macrophages as described above. In other words, the presence of an increase of these cell types is a biomarker that indicates that treatment with the primary cell derived biologic will be effective. This method can be used to screen for patients for whom treatment with the primary cell derived biologic would not be successful so that these patients can seek other alternatives. This method can use automated means for predicting the treatment outcome, such as, but not limited to, various assays or immunoassays (ELISA, radioimmunoassays) and high-throughput methods.

The present invention provides a method of immune prophylaxis, including the steps of administering an effective amount of the primary cell derived biologic, and preventing immune suppression. Immune prophylaxis is the prevention of the immune system from being suppressed. The primary cell derived biologic actively turns on all parts of the immune system, specifically by maturing immature dendritic cells, activating naïve T cells, the resulting mature dendritic cells activating the naïve T cells, protecting the activated naïve T cells from apoptosis (especially when administered before performing chemotherapy or irradiation), differentiating the naïve T cells into memory and effector T cells, and activating regional lymph nodes so that the immune system does not become suppressed. Each of these steps are as described above. If a patient is prone to immune suppression due to biological factors, this patient can be given IRX-2 preemptively to prevent their immune system from becoming depressed. For example, if a patient has certain genetic factors that predispose them to developing cancer, IRX-2 can be administered so that in the event that an immune target such as cancer does become present, the immune system will be ready to attack the immune target.

The present invention also provides for a method of immune restoration, including the steps of administering an effective amount of the primary cell derived biologic, and restoring the immune system of a patient. Patients who have a suppressed immune system benefit from IRX-2 treatment and have their immune system restored to normal or higher levels of function. More specifically, the immune system is restored by maturing immature dendritic cells, activating naïve T cells, the resulting mature dendritic cells activating the naïve T cells, protecting the activated naïve T cells from apoptosis, modifying populations of B and T cells in blood, activating regional lymph nodes, infiltrating an area adjacent to an immune target with T helper and B cells, and infiltrating the immune target with T killer cells and macrophages. Each of these steps are as described above. Multiple arms of the immune system are turned on by the administration of IRX-2, and thus, the immune system can now respond to immune targets. For example, tumors and other immune targets tend to down-regulate various immune components needed to attack that immune target. Immune targets have a protective effect on themselves so that they are not attacked by the immune system. Furthermore, the dendritic cells of the immune suppressed patients become tolerant of the presence of the immune target. These immune targets are susceptible to attack, however, once the immune system has been unsuppressed. IRX-2 breaks the tolerance of the dendritic cells to the immune target, and activates each of the arms of the immune system as described above in order to overcome all of the protective effects of the immune target. The effect of the primary cell derived biologic on dendritic cells is described in U.S. Pat. Nos. 6,977,072 and 7,153,499.

The present invention also provides for a method of treating a tumor, including the steps of administering an effective amount of a primary cell derived biologic, modifying populations of B and T cells in blood, activating regional lymph nodes, peritumorally infiltrating the tumor with T helper and B cells, intratumorally infiltrating the tumor with T killer cells and macrophages, and treating the tumor. Each of these steps are as described above. IRX-2 is shown below in the Examples to treat tumors in various stages of cancer as evidenced by softening of the tumor, reducing pain caused by the tumor, reducing the size of the tumor, fragmentation of the tumor, necrosis of the tumor, and fibrosis of the tumor. In essence, IRX-2 unsuppresses each of the arms of the immune system so that a tumor can effectively be treated and cancer eradicated from a patient.

The present invention further provides for a method of preventing tumor escape, including the steps of, administering an effective amount of a primary cell derived biologic, producing an immune regression of a tumor by modifying populations of B and T cells in blood, activating regional lymph nodes, peritumorally infiltrating the tumor with T helper and B cells, intratumorally infiltrating the tumor with T killer cells and macrophages, and preventing tumor escape. Each of these steps are as described above. Many tumors are invulnerable to the immune system and send out signals to suppress the immune system. Since the immune system is completely unsuppressed by IRX-2, the tumors do not escape from the immune system and metastasize. Importantly, none of the patients in the Examples below experienced a recurrence of tumors after IRX-2 treatment. Thus, IRX-2 effectively prevents tumor escape.

Overall, IRX-2 unsuppresses each of the different arms of the immune system to attack various immune targets. Any immune incompetent disease state (cancer, AIDS, and others as previously described above) can now be reversed by unsuppressing the immune system through IRX-2. IRX-2 functions as a “symphony” rather than just a single “instrument” in that the specific combination of cytokines of IRX-2 effect multiple parts of the immune system, as opposed to prior art therapeutics which, while being combinations of components, only work on a single part of the immune system. Each part of the immune system is a gatekeeper of one effect experienced by IRX-2 administration. Each of these parts of the immune system is required in order to attack an immune target. In other words, as shown in FIG. 17, immature dendritic cells must become mature in order to activate naïve T cells. Production of naïve T cells also must be induced so that they can be presented with antigen by the mature dendritic cells. Both the naïve T cells and the dendritic cells must migrate to the regional lymph node in order for antigen to be presented to the naïve T cells by the dendritic cells. Once activated, the naïve T cells must be protected from apoptosis so that they can differentiate into killer T cells and attack the immune target. B cells also must become macrophages to aid in attacking the immune target. Administration of IRX-2 allows for the performance of each of these functions and provides a healthy and functioning immune system that is ready to attack any immune target.

Dosing of the primary cell derived biologic in vivo is the same as the vaccine+IRX-2 or IRX-2 alone immunotherapy disclosed in the previously mentioned patents related to IRX-2. IRX-2 is preferably injected perilymphatically over a 10 day regimen at 115 Units per injection, but can also be injected with other methods further described below. Alternatively, other reginous tanks used wherein the IRX-2 is administered intermittently. For example, it can be administered three days a week or five out of seven days a week. As shown below, IRX-2 inhibited apoptosis over a range of concentrations: from 1:1 to 1:10 dilution of the IRX-2 liquid (i.e. dilution of the IRX-2 in the media in which it was grown).

Preferably, the IRX-2 is injected around lymphatics that drain into lymph nodes regional to a lesion, such as a tumor or other persistent lesions being treated. Perilymphatic administration into the lymphatics, which drain into the lymph nodes, regional to the lesion, such as a cancer, is critical. Peritumoral injection has been associated with little response, even progression and is thus contraindicated. A ten (10) day injection scheme is optimal and a twenty (20) day injection protocol, while effective clinically, tends to reduce TH1 response and likely shifts towards a less desirable TH2 response as measured by lymphoid infiltration into the cancer. Bilateral injections are effective. Where radical neck dissection has occurred, contralaterial injection is effective.

The compounds of the present invention (including IRX-2) are administered and dosed to promote protection from apoptosis as well as optimal immunization either to exogenous or endogenous antigen, taking into account the clinical condition of the individual patient, the site and method of administration, scheduling of administration, patient age, sex, and body weight. The pharmaceutically “effective amount” for purposes herein is thus determined by such considerations as are known in the art. The amount is preferably effective to protect T cells from apoptosis. The amount is also preferably effective to promote immunization, leading to, e.g., tumor reduction, tumor fragmentation and leukocyte infiltration, delayed recurrence or improved survival rate, or improvement or elimination of symptoms.

In the methods of the present invention, the compounds of the present invention can be administered in various ways, although the preferred method is by perilymphatic injection. It should be noted that the compounds can be administered as the compounds themselves or as a pharmaceutically acceptable derivative and can be administered alone or as an active ingredient in combination with pharmaceutically acceptable carriers, diluents, adjuvants and vehicles. The compounds can also be administered intra- or subcutaneously, or peri- or intralymphatically, intranodally or intrasplenically or intramuscularly, intraperitoneally, and intrathorasically. Implants of the compounds can also be useful. The patient being treated is a warm-blooded animal and, in particular, mammals including man. The data presented shows activity of the IRX-2 on humans or cells derived from humans, and therefore the data herein is all directly relevant and applicable to humans. The pharmaceutically acceptable carriers, diluents, adjuvants and vehicles as well as implant carriers generally refer to inert, non-toxic solid or liquid fillers, diluents or encapsulating material not reacting with the active ingredients of the invention.

The doses can be single doses or multiple doses over a period of several days, although preferably a 10 day injection scheme is used. When administering the compound of the present invention, it is generally formulated in a unit dosage injectable form (e.g., solution, suspension, or emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

Proper fluidity can be maintained, for example, by the use of a coating such as lecithin, by the maintenance of the required particle size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, can also be used as solvent systems for compound compositions. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it is desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the compounds.

Sterile injectable solutions can be prepared by incorporating the compounds utilized in practicing the present invention in the required amount of the appropriate solvent with several of the other ingredients, as desired.

A pharmacological formulation of the present invention can be administered to the patient in an injectable formulation containing any compatible carrier, such as various vehicles, additives, and diluents; or the compounds utilized in the present invention can be administered parenterally to the patient in the form of slow-release subcutaneous implants or targeted delivery systems such as monoclonal antibodies, vectored delivery, iontophoretic, polymer matrices, liposomes, and microspheres. Examples of delivery systems useful in the present invention include those disclosed in: U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many other such implants, delivery systems, and modules are well known to those skilled in the art.

The invention is further described in detail by reference to the following experimental examples. These examples are provided for the purpose of illustration only, and are not intended to be limiting unless otherwise specified. Thus, the present invention should in no way be construed as being limited to the following examples, but rather, be construed to encompass any and all variations which become evident as a result of the teaching provided herein.

### EXAMPLES

### Materials and Methods

All steps relating to cell culture are performed under sterile conditions. General methods of cellular immunology not described herein are performed as described in general references for cellular immunology techniques such as Mishell and Shiigi (Selected Methods in Cellular Immunology, 1981) and are well known to those of skill in the art.

### Preparation of Primary Cell Derived Biologic (IRX-2)

The method of making the primary cell derived biologic is generally described in U.S. Provisional Patent Application No. 61/044,674. Mononuclear cells (MNCs) are purified to remove contaminating cells by loading leukocytes onto lymphocyte separation medium (LSM) and centrifuging the medium to obtain purified MNCs with an automated cell processing and washing system. The MNCs are then stored overnight in a FEP lymphocyte storage bag. An induction mixture of the MNCs is stimulated with a mitogen, preferably phytohemagglutinin (PHA), and ciprofloxacin in a disposable cell culture device and a primary cell derived biologic is produced from the MNCs. The mitogen is removed from the induction mixture by filtering and tangential flow filtration mode, and then the induction mixture is incubated. The induction mixture is clarified by filtering to obtain a primary cell derived biologic supernatant. Finally, the primary cell derived biologic supernatant is cleared from DNA and adventitious agents by applying anion exchange chromatography and 15 nanometer filtration and optionally further inactivation by ultraviolet-C (UVC). The final product can then be vialed and stored for future administration to a patient.

### Example 1

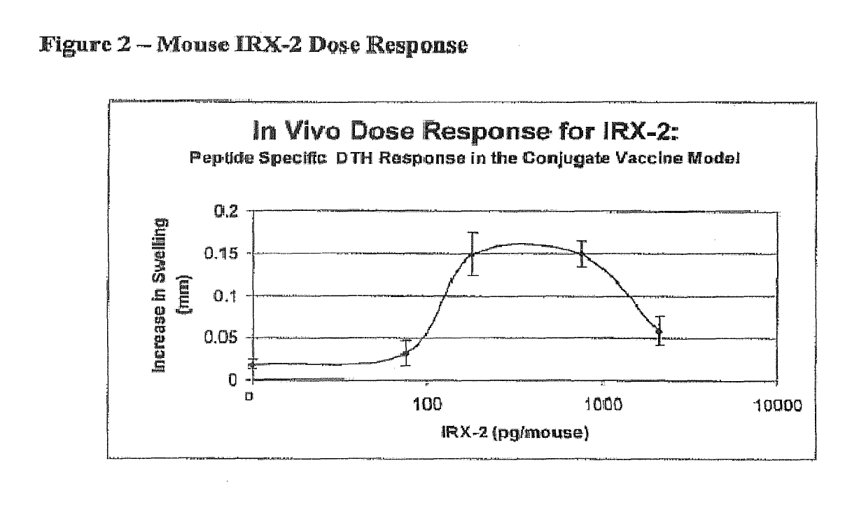

The selection of the dose and schedule for the IRX-2 regimen to be used in experiments was based on studies conducted by IRX Therapeutics. The IRX Therapeutics study was performed in mice immunized with prostate specific membrane antigen (PSMA) peptide conjugate and assessed as increase in footpad swelling. FIG. 2 shows these data and the characteristic “bell-shaped” curve.

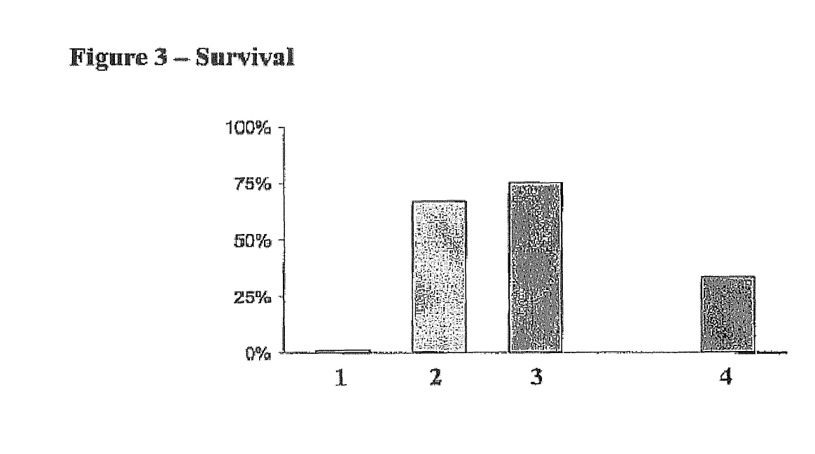

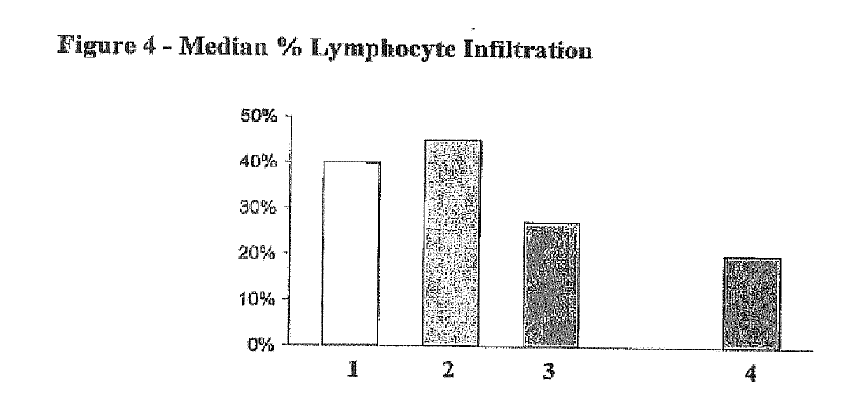

The study was performed in four groups of patients, as shown in Table 1 below. The graph of tumor lymphocyte infiltration and survival for these groups are presented in FIGS. 3 and 4.

TABLE 1

| | | | | | Cumulative |
|---|---|---|---|---|---|
| | | Dose of IRX-2 | Injections/ | | Dose of |
| Regimen | N | injection (Units) | day | # days | IRX-2 (Units) |
| 1 | 4 | ~38 U | 1 | 10 | 380 U |
| 2 | 15 | ~115 U | 1 | 10 | 1,150 U |
| 3 | 10 | ~115 U | 2 | 20 | 4,600 U |
| 4 | 6 | ~660 U | 2 | 20 | 26,400 U |

In this study, maximum lymphoid infiltration was achieved for patients treated with the 10 days of 115 U IL-2 equivalence/day. Survival was poor in the four patients who received the lowest dose (regimen 1). Similarly, poorer survival was noted in six patients treated with the highest dose. While survival appeared to be comparable for regimens 2 and 3, regimen 2 patients experienced the most significant histological response as measured by lymphoid infiltration.

The dose of IRX-2 to be studied further was subsequently selected as intermediate between the two most active doses investigated (regimens 2 and 3), a dose clearly adequate to achieve significant histological changes in tumor and lymph nodes. Based upon the additional inconvenience of 20 versus 10 days of treatment and the lesser lymphoid infiltration in the patients who received the higher IRX-2 dose, a 10-day injection protocol with bilateral injection (approximately 2300 U total of IRX-2) was selected for the further studies discussed below.

### Example 2

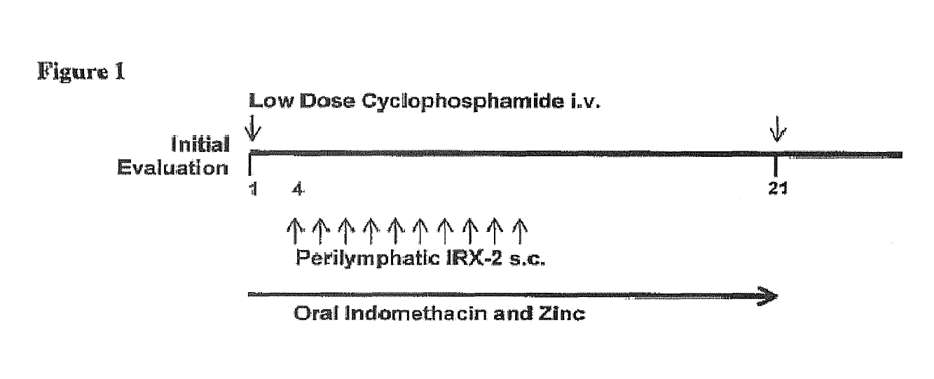

A study of the IRX-2 protocol was performed in H&NSCC patients prior to surgery and/or radiotherapy and/or chemoradiotherapy as described in FIG. 1. IRX-2 was administered bilaterally at 115 Units/site. Twenty seven patients were treated; their demographics summarized in Table 2.

TABLE 2

| Number of treated patients | 32 |
|---|---|
| Median age (range) | 66 (34-86) |
| M:F ratio | 25:7 |
| KPS range | 70-100 |
| Patient Characteristics | |
| Oral | 15 |
| Larynx | 13 |
| Other | 4 |
| Stage at Diagnosis | |
| I | 1 |
| II | 5 |
| III | 10 |
| IV | 15 |
| NA | 1 |
| | No. (%) |
| Stage of primary tumor | |
| T1 | 1 (4) |
| T2 | 15 (56) |
| T3 | 6 (22) |
| T4 | 5 (19) |
| TX | 0 |
| Nodal stage | |
| N0 | 5 (19) |
| N1 | 8 (30) |
| N2 | 14 (52) |
| N3 | 0 |
| NX | 0 |

Radiological studies (CT or MRI) were performed at the onset and prior to surgery and reviewed centrally (Perceptive, Waltham, Mass.). Blood was analyzed centrally (Immunosite, Pittsburgh, Pa.) at onset and prior to surgery for various leukocyte populations (Table 3 and 4). Surgical samples were sent to a central reference laboratory (Phenopath, Seattle, Wash.) for evaluation of the histological changes and performance of immunohistochemistry for various leukocyte markers (Table 5). Appropriate laboratory and clinical measurements were performed to assess toxicology and symptomatic improvement throughout disease-free and overall survival continue to be monitored.

### Clinical Results:

Three patients had objective tumor responses (2PR; 1MR). Four patients showed radiological responses (>12.5% reduction); five patients (N2C, N2C, N1, N1, N1) were down-staged as nodes detected as tumor-positive at the sites and centrally were shown to be negative in the surgical specimens. Four tumors softened (a positive sign), 14 patients had symptomatic improvement/reduced pain and tenderness, improved swallowing, and less bleeding. Treatment related side effects were generally mild (grade I or II) and infrequent including nausea, vomiting, dry mouth, constipation, injection site pain, headache, myalgia, anemia, and contusion. A single example of dyspepsia grade III was observed. Disease-free and overall-survival are being followed. Most patients have cleared one year and survival curves closely parallel those previously observed by Applicant in studies at the National Cancer Institute of Mexico and appear better than case-matched U.S. and Mexican controls.

### Example 3

Heparinized blood was collected for immunophenotyping studies to determine numbers of immune cell subsets including B, T, NK, and T naïve, T memory, and T effector cells. Fluorescently tagged monoclonal antibodies to the indicated cell surface markers (or corresponding isotope control) were used to stain fresh, unfractionated whole blood.

The stained and fixed samples were then acquired and analyzed by multi-parameter flow cytometry using a Beckman Coulter FC500 flow cytometer and CXP TM analysis software. Enumeration of absolute T lymphocyte subsets using this single platform (flow cytometry only) method that employs Flow Count TM beads has been demonstrated to be more accurate than dual (hematology instruments and flow cytometry) platform techniques (Reimann et al., 2000). Table 3 below presents a list of the immune markers analyzed by ImmunoSite and their role in an immunization.

TABLE 3
Immune Markers Analyzed & Role in Immune Response

| Cell | Marker | Role |
|---|---|---|
| T cell | CD3 | Mediates cellular immunity |
| B cell | CD3− CD19+ CD14− | Mediates humoral immunity |
| Helper T cell | CD3+ CD4 | Makes cytokines, provides |
| | | B cells “help” |
| Cytotoxic T cell | CD3+ CD8 | Kills tumor cells |
| Naïve T cell (T_{N}) | CD3+ CD45RA+ CCR7+ | Antigen naïve or very early |
| | | post-primary stimulation; |
| | | lymph node homing ability |
| Central Memory T cell | CD3+ CD45RA− CCR7+ | Long-lived memory cell, |
| (T_{CM}) | | low effector function; |
| | | homes to lymph nodes |
| Effector Memory T cell | CD3+ CD45RA− CCR7− | Intermediate effector |
| (T_{EM}) | | function; shorter half-life in |
| | | vivo; seeds tissues/tumors |
| | | over lymph nodes |
| Effector T cell (T_{EMRA}) | CD3+ CD45RA+ CCR7− | Highest effector function |
| | | (e.g. cytolysis); localizes |
| | | best to tissues/tumor |

For the purposes of the present invention, only the cell populations directly relevant to evaluating the hypothesis of whether an immunization occurred or not are discussed herein.

The developmental pathways for T lymphocytes, especially CD8+ T cells, have been intensively studied over the last decade with a particular focus on CD8+ T cells since they are most closely associated with effective anti-tumor immunity. Both CD4+ helper T cells and CD8+ cytotoxic T cells can be subdivided into reciprocal CD45RA+ and CD45RO+ subpopulations. CD45RA+ cells have previously been termed naïve T cells; however, more recent work indicates that these T cells in blood comprise naïve T cells as well as more fully differentiated effectors often termed T_{EMRA} (Lanzavecchia, 2005; Kaech, 2002). CD45RO+(CD45RA−) memory T cells can also be subdivided into T central memory (T_{CM}) and T effector memory (T_{EM}). These sub-classifications are based upon surface expression of additional markers including CCR7 (Sallusto, 1999; Tomiyama, 2004). The developmental pathways of these various T cell subsets and their lineage relationships remain complex. The data and tests for significance are presented in Table 4 below.

TABLE 4
Summary of Immunology Assessments & Tests of Significance

| | | | | Baseline | | | | |
|---|---|---|---|---|---|---|---|---|
| | | Mean | | to Day | | Degrees | | |
| Cell | | cells/ | Std | 21 | Std | of | | P |
| population | N | mL³ | Dev | Difference | Dev | Freedom | T value | value |
| Baseline | | | | | | | | |
| Lymphocyte | 25 | 1177.5 | 442.4 | −69.6 | 260.7 | 24 | −1.33 | 0.1946 |
| Gate | | | | | | | | |
| B cell | 18 | 275.4 | 132.2 | −74.3 | 74.8 | 17 | −4.22 | 0.0006 |
| Helper T cell | 25 | 817.0 | 330.7 | −65.4 | 184.0 | 24 | −1.78 | 0.0884 |
| Cytotoxic T | 25 | 351.9 | 193.3 | −4.4 | 87.9 | 24 | −0.25 | 0.8061 |
| cell | | | | | | | | |
| Naïve T cell | 25 | 55.6 | 89.8 | −38.2 | 76.9 | 24 | −2.49 | 0.0203 |
| Central | 25 | 56.9 | 84.5 | −22.8 | 48.6 | 24 | −2.34 | 0.0280 |
| Memory T cell | | | | | | | | |
| Effector | 25 | 689.0 | 354.7 | 41.2 | 223.4 | 24 | 0.92 | 0.3651 |
| Memory T cell | | | | | | | | |
| Effector | 25 | 395.0 | 250.2 | −35.2 | 132.7 | 24 | −1.33 | 0.1968 |
| Memory RA T | | | | | | | | |
| cell | | | | | | | | |

Consistent with the hypothesis that IRX-2 acts on both T cells and DC's to foster activation, maturation, and enhance endogenous tumor antigen presentation to naïve T cells, it was observed that the naïve T cell population (CD3+ CD45RA+ CCR7+) decreased between baseline and Day 21. Naïve T cells are initially activated by recognition of antigen when presented on the appropriate major histocompatibility complex (MCH) molecules by mature DC's. The subsequent steps of generating T cell memory and full effector function are not perfectly defined, but it is clear that different subpopulations of T cells as defined by several markers, i.e. CD45RA/RO and CCR7 have distinct functional properties. For example, CCR7 expression confers the ability of the T cell to home to lymph nodes where the most effective anti-tumor priming occurs.

A significant decline was observed in the naïve T cell population (CD3+ CD45RA+ CCR7+) with population levels of 55.6 cells/mL³ at baseline falling to 17.4 cells/mL³ at Day 21 (p=0.02). A loss of naïve T cells results from those cells finding and being stimulated by their respective cognate antigen and the differentiating into an alternative functional population, either of the two memory or full effector populations.

In addition, the central memory T cell population (CD3+ CD45RA− CCR7+) with the CCR7+ conferred lymph node homing propensity, fell from 56.9 cells/mL³ at baseline to 34.1 cells/mL³ at Day 21 (p=0.028). This too is an indicator that immunization to tumor antigens is taking place in response to IRX-2 therapy. Studies show that the T_{CM} population of T cells represents the earlier, more “stem-like” memory population that upon re-stimulation, preferentially homes to the lymph node where it can gain more effector, e.g. cytolytic function. The significant decline seen in this population is consistent with these T_{CM} cells exiting the bloodstream and migrating to the draining lymph nodes where they will be further activated.

After an immunization, one would expect other immune cells to be enlisted in the attack on the antigen-bearing offender. Further support to the immunization hypothesis was observed in that a significant drop (p<0.01) in B cells was observed. B cells are recruited into lymph nodes where they are exposed to antigen and then exit to be found in the tumor where they presumably produce antibodies capable of attacking the tumor directly or supporting antibody-dependent cellular cytotoxicity (ADCC).

The statistically significant changes and trends observed herein strongly show that an immunization of naïve T cells is occurring due to IRX-2 administration. As no other primary interventions were observed in these patients, it is unlikely that these changes occurred at random.

The hypothesis that IRX-2 treatment induces immunization to autologous tumor antigens is also supported by Applicant's published information on H&NSCC lymph node response following IRX-2 treatment as compared to non-randomized normal and H&NSCC control patients (Meneses, 2003). The salient lymph node response features associated with IRX-2 treatment were nodal replenishment and lymphocyte expansion, particularly T lymphocytes, which were shown to be depleted in the lymph nodes of untreated H&NSCC patients (Verastegui, 2002). Nodal expansion that occurs during an immunization presumably due to IRX-2 was also observed to be associated with a reversal of sinus histiocytosis, an apparent dendritic cell functional defect. These changes are consistent with an immunization. A prior study confirms that immunization to tumor antigen occurs at the level of the regional lymph node, not the tumor itself (Maass, 1995).

### Histology

When an immunization occurs in lymph nodes, the new killer memory T cells are thought to develop and then exit the nodes through blood vessels, and flow into tissues to patrol for the antigenic target (i.e. the immune target). If the antigenic target is identified, the killer memory T cell will infiltrate the tissue to kill the target. When a cellular immune response is initiated, other immune cells are recruited to participate in the kill and clean-up process.

T lymphocyte infiltration into tumors, particularly of CD45RO+ CD8+ T cells, is evidence of an immunization to tumor antigens and that such infiltration correlates with improved survival in a variety of cancers including H&NSCC, melanoma, colorectal, and ovarian (Wolf, 1986; Pages, 2005; Galon, 2006.

It was hypothesized herein that an IRX-2 induced immunization in lymph nodes would result in lymphocytic infiltrate in the tumor and tumor disruption and the presence of specific immune cells in the tumor would provide evidence of an anti-tumor immune response. It was also hypothesized that an immune response to the tumor would be evidenced by diffuse lymphocytic infiltrate, spanning the tumor's peripheral area to its intratumoral area.

Formalin fixed paraffin embedded blocks or unstained slides from primary tumor biopsy and resection specimens were submitted by the clinical sites to PhenoPath Laboratories (Seattle, Wash.) for hematoxylin and eosin (“H&E”) and immunohistochemistry staining (“IHC”). Paired samples from 26 IRX-2 study subjects were submitted, 25 were evaluable, and one surgical specimen had no histological evidence of tumor. Two ad-hoc comparator groups of surgical specimens were collected at the end of the study for H&E comparison: 25 surgical specimens from MD Anderson, and 10 surgical specimens from Stony Brook Health Sciences Center, randomly selected from untreated H&NSCC surgical specimens.

Immunohistochemistry staining was performed only on the IRX-2 treated samples to determine the presence of immune markers in the tumor. Their markers are listed in Table 5.

TABLE 5
Immune Markers Analyzed by IHC

| Cell | Marker | Role in Immune Response |
|---|---|---|
| T cell | CD3 | Mediate cellular immunity |
| B cell | CD20 | Produce antibody |
| Helper T cell | CD4 | Make cytokines; help B cells |
| Cytotoxic T cell | CD8 | Kill tumor |
| Plasma cell | CD138 | Produce antibody |
| Macrophage | CD68 | Assist T cell and kill tumor |
| Naïve/Effector T cell | CD45RA+ | Naïve/Effector T cell |
| Memory T cell | CD45RO (RA−) | Antigen committed T cell |

The presence of IHC stained markers was evaluated under low power and graded using a prospectively defined 0-100 mm visual analog scale (VAS), where 0 represented 0% presence and 100 represented 100% of cells staining positive for the marker. The peroxidase reaction used to highlight the marker overestimates the area or density of lymphocyte infiltration as compared to H&E staining, thus making IHC-based density determinations unreliable, but IHC remains useful for elucidating the relative relationships between and among cell types.

### H&S Studies: Methods and Analyses

Three analyses were performed comparing the H&E stained slides. Two analyses were blinded feature extractions from the 25 IRX-2 treated and 25 untreated surgical specimens from M D Anderson, one for tumor features and one for immune response features. The third analysis was an identical but unblended immune response feature extraction from the 10 H&E stained slides from Stony Brook. In each case, features were extracted and quantified using a VAS on case report forms.

Two assessments were made for each of the immune response features, the first assessment was the overall presence of the marker across the entire surgical specimen and the second was to the degree to which the location of the infiltrate was peripheral or intratumoral.

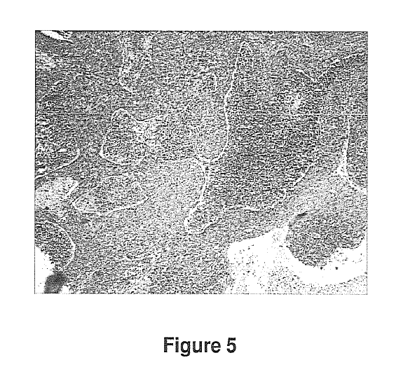

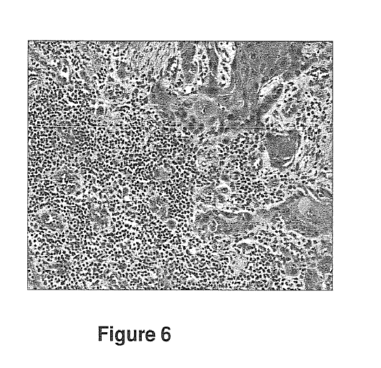

An overall assessment was made taking into account: lymphocyte infiltration, its density, its balance between tumor and infiltration, and other features that comprise the gestalt impression of the tumor. The other sub-features include the extent of fibrosis and necrosis, suggesting where tumor was but is no longer and in the case of well differentiated squamous cell cancer, a concentration of keratin pearls with minimal or not tumor surrounding it is another sign of tumor destruction. An “Active Immunologic Response” includes lymphoid infiltration evidence of damage created by the immune system, and the degree to which tumor is no longer viable and disrupted—in short the extent and process by which the host is combating the tumor. An example of the lymphocyte infiltration sub-feature of the “Active Immune Response” is presented in FIGS. 5 and 6.

One of the dominant sub-features on the Active Immune Response variable is the localization and intensity of the lymphocyte infiltration (LI) that are observed in patients treated with IRX-2. Surgical specimens demonstrating this reaction in both IRX-2-treated patients and the ad-hoc comparator groups demonstrated marked increases in the density of overall LI, peritumoral LI, and intratumoral LI.

Based on the pre-specified critical point of 50 mm or greater on the VAS, the analysis showed different Active Immunologic Response rates among the three groups of surgical specimens as showed in Table 6 below.

TABLE 6

| | | | Active Immune |
|---|---|---|---|
| Group | Patient w/AIR | Total Patients | Response Rate |
| 1. IRX-2 Treated | 11 | 25 | 44.0% |
| 2. MD Anderson | 6 | 1 | 24.0% |
| 3. Stony Brook | 1 | 10 | 10.0% |

The increase in the frequency of those patients demonstrating an Active Immune Response went from 20% in the pooled M D Anderson and Stony Brook groups to 44% in the IRX-2 treated group (p<0.05 by Chi square test).

Determination of Peritumoral Vs. Intratumoral LI

The location of immune cells in the tumor was also evaluated. It was hypothesized herein that an active anti-tumor immune response would include lymphycytic infiltrate that expanded from the peripheral area to include the intratumoral area.

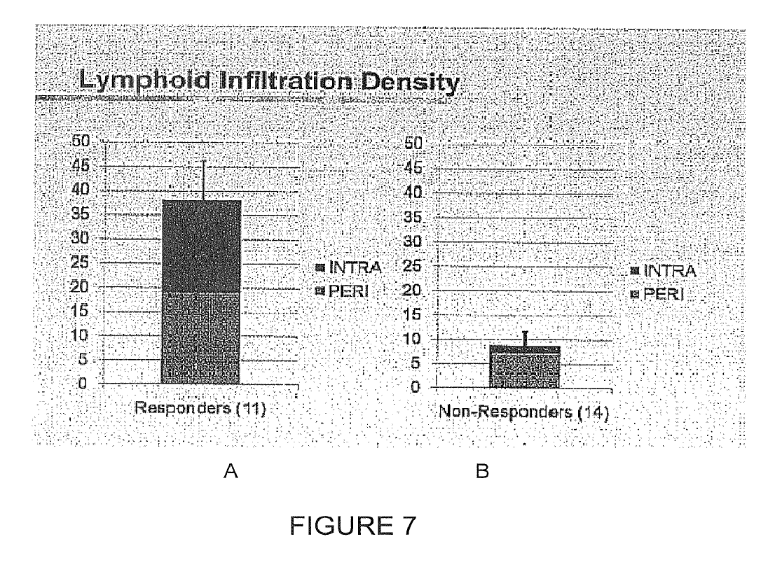

Based upon the VAS analysis for Active Immune Response in the IRX-treated patients, 11 showed intense reactions (≧50, termed responders) and 14 showed less intense reactions (<50, termed non-responders). A comparison of the LI of these two groups is shown in FIGS. 7A and 7B.

As can be seen, the responders showed a marked increase in LI (both area and density) of the typical section and compared to the non-responders, the increase in intratumoral LI is proportionally much greater than the peritumoral change.

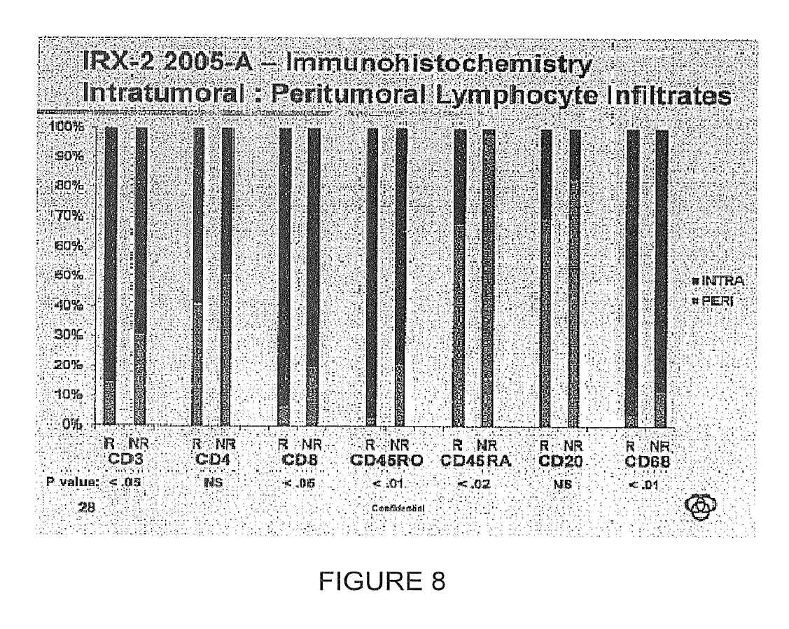

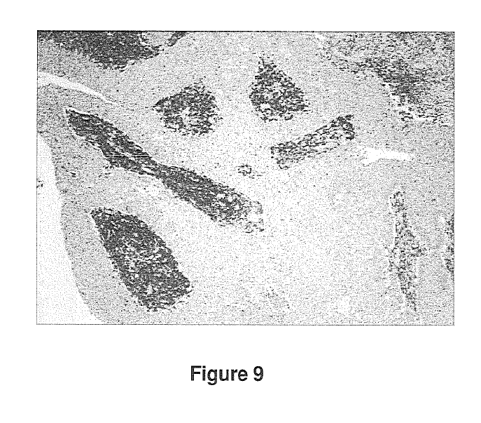

Immunohistochemistry for the location of various markers helps clarify which cells dominate in each region. FIG. 8 shows these results. The peritumoral infiltrate, representing approximately 25% of the LI in the specimen was dominated by CD45RA+, CD3+, CD4+ T lymphocytes and CD20+ B lymphocytes. Whereas the intratumoral infiltrate, representing approximately 75% of the LI in the specimen, was dominated by CD45RO+, CD3+ and CD8+ lymphocytes (i.e. the “killer” effector T cell phenotype) and CD68+ macrophages. FIG. 9 provides a pictoral example of IHC staining fro CD45RO+ memory T cells in an IRX-2 treated surgical specimen.

The strongest support for this immunization hypothesis derives from the examination of lymphocyte infiltration for infiltration in and around the tumor and the picture of tumor rejection indicating necrosis, fibrosis, and reduced tumor. The rejection patterns are characteristic for both humoral and cellular immunity with increased B lymphocytes and activated macrophages within the tumor, respectively.

### Example 4

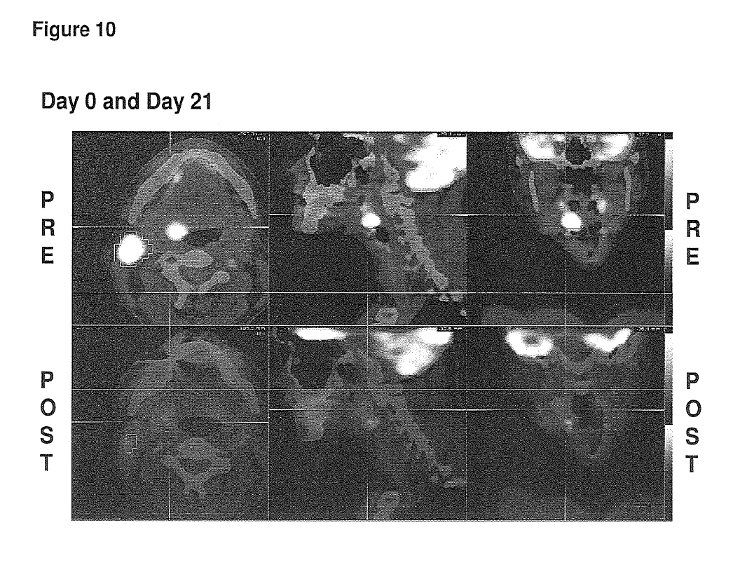

In one patient, fused FDG PET/CT scans were compared at day 0 and day 21, as shown in FIG. 10. Total glycolitic activity and volume were measured and are shown in Table 7.

TABLE 7

| | | |
|---|---|---|
| Baseline | Day 21 | % Change |

Total Glycolytic Activity

| | | | |
|---|---|---|---|
| Tumor | 68.91 | 31.36 | −54.49% |
| Node 1 | 72.54 | 4.97 | −93.15% |
| Node 2 | 14.35 | 3.15 | −78.05% |
| | 155.80 | 39.48 | −74.66% |

Volume

| | | | |
|---|---|---|---|
| Tumor | 12.16 | 7.33 | −39.72% |
| Node 1 | 9.46 | 1.44 | −84.78% |
| Node 2 | 2.28 | 1.24 | −45.61% |
| | 23.90 | 10.01 | −58.12% |

### Example 5

Previously, the criteria for histopathology of a biopsy versus a tumor specimen (Meneses) were that the tumor was reduced overall, fragmentation of the tumor occurred, and there was increased lymphocyte infiltration (LI). According to the present invention, there are new criteria presented herein for a treated tumor versus a control tumor, namely tumor disruption with necrosis and fibrosis, and increased LI that is greater intratumorally than peritumorally. Table 8 below summarizes various findings of cytokine treatment on H&NSCC. Importantly, IRX-2 is shown to work on all arms of the immune system whereas other multiple component cytokine therapeutics do not. MULTIKINE (Cel-Sci) includes multiple cytokines in its formulation; however, its effect is a single one on the tumor itself, not on the immune system.

TABLE 8

| | Treated | Control | |
|---|---|---|---|
| De Stefani | Tumor | Control | ↑ LI, ↑ necrosis, ↑ fibrosis |
| rIL-2 | | tumor | |
| Meneses | Tumor | Biopsy | ↑ LI, ↓ tumor, ↑fragmentation |
| IRX-2 | | | |
| Feinmesser | Tumor | Biopsy | ↑ LI, ↓ tumor |
| Multikine | | | |
| Timar | Tumor | Control | ↑ LI, No ↓ tumor or fragmentation |
| Multikine | | tumor | |
| IRX Therapeutics | Tumor | Biopsy | ↑ LI - small tumor, ↑fragmentation |
| | Tumor | Control | ↑ LI, ↑ fibrosis |
| | | tumor | |

### CONCLUSION

This study confirms and extends Applicant's prior observations concerning the ability of the IRX-2 regimen to have significant biological activity on patients with squamous cell head and neck cancer treatment prior to surgery. The present study confirms that the treatment is safe with few adverse events attributed to the regimen. In fact, those patients who showed evidence of histopathologic changes of lymphocyte infiltration had the majority of symptom improvements like reduced pain and tenderness, improved breathing and phonation, and softening of the tumor (as sign of dissolution). Three patients were adjudged to have clinical responses (2PRs, 1MR). Overall survival data and recurrence free survival while immature are encouraging and similar in degree and profile to Applicant's previous study. Notable is that no deaths occurred due to recurrence in the first 12 months of follow up. All deaths to date but one are in the non-responder group.

The most compelling data are those associated with the mechanism of action studies. It was observed that declines of B lymphocytes and two T cell subsets associated with initial immunization and lymph node homing. No increases in memory/effector cell were observed in blood; however, this is explainable based upon the traffic patterns of T cells which occur with an immunization. Notably no increase in T regs was observed.

Applicant's prior studies showed that patients responding to the IRX-2 regimen show increase of uninvolved lymph nodes proximal to the tumor, replenishment of depleted T lymphocyte areas and the picture of activation as occurs with antigen. Thus, lymphocytes are trafficking via blood and lymphatics to the regional lymph nodes where they are presumably immunized to autologous tumor antigens. As shown herein, they then leave the lymph node and travel by blood to the tumor where they infiltrate in and around the tumor and correlate with evidence of tumor destruction (necrosis, fibrosis, and tumor reduction). In the patients showing this reaction, the increases in lymphocyte infiltration involves predominantly CD3+ CD4+ CD45RA+ T cell populations and CD20+ B lymphocytes around the tumor periphery and CD3+ CD8+ CD45RP+ T lymphocyte populations and macrophages within the tumor. The changes within the tumor are greater than these in the periphery. This mechanism is generally shown in FIG. 17.

Notably, untreated patients show such a reaction only occasionally (20%) and while significantly less frequently than patients treated with the IRX-2 regimen (44% vs. 20%) the presence of the reaction in controls represent a new biomarker for predicting favorable outcome.

The picture is an integrated one clinically, radiologically, pathologically, and immunologically and provides ample evidence for an immunization to autologous tumor antigen. IRX-2 is shown to activate all arms of the immune system to provide a total restoration of immune function and ability to attack immune targets.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology which has been used is intended to be in the nature of words of description rather than of limitation.

Obviously, many modifications and variations of the present invention are possible in light of the above teachings. It is, therefore, to be understood that within the scope of the appended claims, the invention may be practiced otherwise than as specifically described.

### REFERENCES

1. Dunn G, et al. Dendritic cells and HNSCC: A potential treatment option? (Review). Oncology Reports 13:3-10, 2005.
2. Egan J E, et al. IRX-2, a novel in vivo immunotherapeutic, induces maturation and activation of human dendritic cells in vitro. J Immunother 30:624-633, 2007.
3. Galon J, et al. Type, density, and location of immune cells within human colorectal tumors predict clinical outcome. Science 313:1960, 2006.
4. Hadden J W, et al. Immunotherapy with natural interleukins and/or thymosin alpha 1, potently augments T-lymphocyte responses of hydrocortisone-treated aged mice. Int J Immunopharm 17(10):821-828, 1995.
5. Hadden J W, et al. Sinc induces thymulin secretion from human thymic epithelial cells in vitro and augments splenocytes and thymocyte responses in vivo. Int J. Immunopharm 17(9):729-733, 1995.
6. Kaech S M, et al. Effector and memory T-cell differentiation: implications for vaccine development. Nature Rev Immunol 2:251, 2001.
7. Lanzavecchia A, et al. Understanding the generation and function of memory T cell subsets. Curr Opin Immunol 17:326, 2005.
8. Maass G, et al. Priming of tumor-specific T cells in the draining lymph nodes after immunization with interleukin-2-secreting tumor cells: Three consecutive stages may be required for successful tumor vaccination. Proc Natl Acad Sci 92:5540, 1995.
9. Mantovani A, et al. Macrophage polarization: tumor associated macrophages as a paradigm for polarized M2 mononuclear phagocytes. Trends in Immunology, 23 (11) 2002.
10. Meneses A, et al. Lymph node histology in head and neck cancer: impact of immunotherapy with IRX-2. Int'l Immunopharm. 3:1083-1091, 2003.
11. Pages F, et al. Effector memory T cells, early metastasis, and survival in colorectal cancer. NEJM 353:2654-66, 2005.
12. Sallusto F, et al. Two subsets of memory T lymphocytes with distinct homing potentials and effector functions. Nature 401:708, 1999.
13. Tomiyama H, et al. Phenotypic classification of human CD8+ T cells reflecting their function: inverse correlation between quantitative expression of CD27 and cytotoxic effector function. Eur J Immunol 34:999, 2004.
14. Verastegui E, et al. Immunological approach in the evaluation of regional lymph nodes of patients with squamous cell carcinoma of the head and neck. Clin Immunol 102:37, 2002.
15. Whiteside T L. Immunobiology and immunotherapy of head and neck cancer. Curr One Reports 3:46-55, 2001.
16. Wolf G T, et al. Lymphocyte subpopulations infiltration squamous carcinomas of the head and neck: correlations with extent and tumor prognosis. Otolaryngol Head Neck Surg 95:145, 1986.

What is claimed is:

1. A method of treating an immune target that is suppressing the immune system and restoring the immune system, including the steps of:
administering an effective amount of a primary cell derived biologic;
modifying populations of B and T cells in blood;
activating regional lymph nodes;
infiltrating and area adjacent to an immune target with T helper and B cells;
infiltrating the immune target with killer T cells and macrophages; and
treating the immune target and restoring the immune system.

2. The method of claim 1, wherein said modifying step is further defined as upregulating or downregulating the populations of B and T cells in blood.

3. The method of claim 2, wherein said modifying step is further defined as modifying populations of naïve T cells and early memory T cells.

4. The method of claim 3, wherein said modifying step is further defined as modifying CD3+, CD45RA+, and CCR7+ naïve T cell populations.

5. The method of claim 4, wherein said modifying step is further defined as differentiating the naïve T cells into memory and effector T cells.

6. The method of claim 5, further including the step of causing central memory T cells to exit the bloodstream and migrate to draining lymph nodes.

7. The method of claim 3, wherein said modifying step is further defined as causing the B cells to be recruited into lymph nodes, exposing the B cells to antigen, migrating the B cells to the immune target, and attacking the immune target.

8. The method of claim 7, wherein said attacking step is further defined as an action chosen from the group consisting of producing antibodies that attack the immune target, and supporting antibody-dependent cellular cytotoxicity.

9. The method of claim 1, wherein said activating step is further defined as enlarging the regional lymph nodes, replenishing lymphocytes, and reversing sinus histiocytosis.

10. The method of claim 1, wherein said infiltrating the area adjacent to the immune target step is further defined as infiltrating the area adjacent to the immune target with CD45RA+, CD3+, and CD4+ T lymphocytes and CD20+ B lymphocytes.

11. The method of claim 1, wherein said infiltrating the immune target step is further defined as infiltrating the immune target with CD45RO+, CD3+, and CD8+ lymphocytes and CD68+ macrophages.

12. The method of claim 1, wherein said infiltrating the area adjacent to the immune target step and said infiltrating the immune target step produce humoral and cellular immunity.

13. The method of claim 1, wherein the primary cell derived biologic is further defined as IRX-2.

14. The method of claim 13, wherein said administering step further includes administering low dose cyclophosphamide prior to administering the IRX-2 and further includes the step of reversing suppression by T regs lymphocytes.

15. The method of claim 14, wherein said administering step further includes administering indomethacin and zinc daily.

16. The method of claim 14, wherein said administering step is further defined as subcutaneously administering IRX-2 daily or intermittently 3 days a week 5 out of 7 days for 5 to 20 days.

17. The method of claim 16, wherein said administering step is further defined as administering 30 to 700 Units of IRX-2 per day.

18. The method of claim 1, further including the step of administering exogenous antigen.

19. The method of claim 1, further including the step of performing surgery, radiotherapy, chemotherapy, or combinations thereof.

20. The method of claim 1, wherein the immune target is a biological condition caused by the group consisting of genetic defects, cancer, infections, malnutrition, burns, AIDS, HIV, chemotherapy, and radiotherapy.

21. A method of inducing immunization in a patient, including the steps of:
administering an effective amount of a primary cell derived biologic;
detecting a change in T and B cells; and
inducing immunization in a patient.

22. A method of destroying a tumor, including the steps of:
administering an effective amount of a primary cell derived biologic;
maturing immature dendritic cells;
activating naïve T cells;
the resulting mature dendritic cells stimulating the naïve T cells;
differentiating the naïve T cells into killer T cells;
directing killer T cells to a tumor; and
destroying the tumor.

23. The method of claim 22, wherein the primary cell derived biologic is further defined as IRX-2.

24. A method of predicting a favorable treatment outcome to cancer treatment, including the steps of:
administering an effective amount of a primary cell derived biologic;
detecting an increase peritumorally of T helper and B cells and intratumorally of T killer cells and macrophages; and
predicting a favorable treatment outcome to cancer treatment.

25. The method of claim 24, wherein said detecting step is further defined as detecting an increase peritumorally of CD45RA+, CD3+, and CD4+ T lymphocytes and CD20+ B lymphocytes and intratumorally of CD45RO+, CD3+, and CD8+ lymphocytes and CD68+ macrophages.

26. The method of claim 24, wherein the primary cell derived biologic is further defined as IRX-2.

27. A method of immune prophylaxis, including the steps of:
administering an effective amount of a primary cell derived biologic; and
preventing immune suppression.

28. The method of claim 27, wherein said preventing step is further defined as maturing immature dendritic cells, activating naïve T cells, the resulting mature dendritic cells activating the naïve T cells, protecting the activated naïve T cells from apoptosis, differentiating the naïve T cells into memory and effector T cells, and activating regional lymph nodes so that the immune system does not become suppressed.

29. The method of claim 28, wherein the primary cell derived biologic is further defined as IRX-2.

30. A method of immune restoration, including the steps of:
administering an effective amount of a primary cell derived biologic; and
restoring the immune system of a patient.

31. The method of claim 30, wherein said restoring step is further defined as maturing immature dendritic cells, activating naïve T cells, the resulting mature dendritic cells activating the naïve T cells, protecting the activated naïve T cells from apoptosis, modifying populations of B and T cells in blood, activating regional lymph nodes, infiltrating an area adjacent to an immune target with T helper and B cells, and infiltrating the immune target with T killer cells and macrophages.

32. The method of claim 31, wherein the primary cell derived biologic is further defined as IRX-2.

33. The method of claim 32, wherein the immune target is a tumor.

34. A method of treating a tumor, including the steps of:
administering an effective amount of a primary cell derived biologic;
modifying populations of B and T cells in blood;
activating regional lymph nodes;
peritumorally infiltrating the tumor with T helper and B cells;
intratumorally infiltrating the tumor with T killer cells and macrophages; and
treating the tumor.

35. The method of claim 34, wherein the primary cell derived biologic is further defined as IRX-2.

36. The method of claim 34, wherein said treating step provides at least one result chosen from the group consisting of softening of the tumor, reducing pain caused by the tumor, reducing the size of the tumor, fragmentation of the tumor, necrosis of the tumor, and fibrosis of the tumor.

37. A method of preventing tumor escape, including the steps of:
administering an effective amount of a primary cell derived biologic;
producing an immune regression of a tumor by modifying populations of B and T cells in blood;
activating regional lymph nodes;
peritumorally infiltrating the tumor with T helper and B cells;
intratumorally infiltrating the tumor with T killer cells and macrophages; and
preventing tumor escape.

38. The method of claim 37, wherein the primary cell derived biological is further defined as IRX-2.
